(19) United States
(12) Patent Application Publication
(10) Pub. No.: US 2023/0173339 A1
(43) Pub. Date: Jun. 8, 2023

(54) AN ORAL-MOTOR MODULAR TRAINING SYSTEM

(71) Applicant: Yehudit Levy, Ashdod (IL)

(72) Inventor: Yehudit Levy, Ashdod (IL)

(21) Appl. No.: 17/920,793

(22) PCT Filed: May 19, 2021
(86) PCT No.: PCT/IL2021/050573

§ 371 Date: Oct. 23, 2022

(30) Foreign Application Priority Data
May 20, 2020 (IL) 274788

(51) Int. Cl. A63B 23/03; A61F 5/58; A63B 24/00

(52) U.S. Cl. CPC A63B 23/032; A61F 5/58; A63B 24/0062; A63B 2220/833

(57) ABSTRACT

The present invention relates to a modular oral motor training system adapted to operate with multiple electromechanical training devices to strengthen the oral musculature, and improve the oral motor and breathe control, thereby improving the speech fluency and pronunciation of persons challenged with speech disorders. The system comprises a base unit adapted to receive one or more replaceable functional training devices, wherein the base unit comprises an electromechanical port through which it supplies electrical power and receives performance level data to/from the one or more functional training devices, according to which the base unit delivers performance feedback to a user of the system.

### FIELD OF THE INVENTION

The present invention is in the fields of oral motor therapy. More specifically, the invention relates to a modular oral motor training system for improving the speech fluency and pronunciation of persons challenged with speech disorders.

### BACKGROUND OF THE INVENTION

In order to speak, one regularly uses different oral members for forming correct speech sounds and pronunciation. For example, pushing a portion of the tongue against a target area of the palate/teeth while exhaling air, or retracting the laryngeal muscles while rounding the lips and exhaling air.

People of all ages who are challenged with developmental speech disorders, or recover neurological injuries may experience weakness of one or more muscles. This weakness possibly results in difficulty with pronouncing words correctly, distorting sounds, difficulty controlling the speech volume, producing sound sounds of reasonable volume, and pitch.

Professional therapists use various existing assessment tools and procedures (e.g., a speech-language pathologist (SLP)) to diagnose contributing factors for speech disorders. Among which isolating specific weak oral muscles to be strengthened, where following assessment, a series of motor exercises are prescribed to a patient, some of which performed at home by the patient and some at the clinic, using assistive training devices. Following one or more training sessions the patient's oral motor is re-assessed, and the therapeutic procedure is being prescribed accordingly.

Some people may be diagnosed with a single cause for their speech disorder. Therefore, they may require extensive training of a single motor capability such as training the mastication muscles with a training device such as a biting device. Yet, many times a person is required to strengthen more than a single motor capability, requiring multiple training devices.

It is, therefore, an object of the present invention to provide a modular oral motor training system, which provides real-time feedback, thereby being suitable for in-home independent training.

It is another object of the present invention, a modular oral motor training system that comprises multiple training modules, thereby enabling more than a single oral motor capability, thus further supporting independent training.

Other objects and advantages of the invention will become apparent as the description proceeds.

### SUMMARY OF THE INVENTION

The present invention is a modular oral motor training system, comprising a base unit adapted to receive one or more replaceable functional training devices, wherein said base unit comprises an electromechanical port through which said base unit supplies electrical power and receives performance level data to/from said one or more functional training devices, according to which said base unit delivers performance feedback to a user of said modular oral motor training system.

According to an embodiment of the invention, each of the one or more functional training devices comprises an operable mechanism, suitable means for assessing its mechanical operation by a user, control and processing hardware for coding the assessed mechanical operation of said operable mechanism by the user into a performance level, and electromechanical means for coupling said one or more functional training devices with said coupling electromechanical port of said base unit, wherein said performance level is transmitted to said base unit for producing a corresponding feedback.

According to an embodiment of the invention, the delivered feedback is selected from the group consisting of: audial feedback, visual feedback, mechanical feedback, or any combination thereof.

According to an embodiment of the invention, the electromechanical port comprises a Pogo Pins connector.

According to an embodiment of the invention, the base unit is cylindrical.

According to an embodiment of the invention, the base unit comprises a communication module.

According to an embodiment of the invention, the one or more functional training devices are selected from the group consisting of: an air-blow training device, a tongue thrust device, a suction device, a mastication training device, a sound production training device, or any combination thereof.

### BRIEF DESCRIPTION OF THE DRAWINGS

FIG. 1 shows an exemplary configuration of a modular oral motor training system, according to an embodiment of the present invention;

FIG. 2 shows a base unit 101, according to an embodiment of the present invention;

FIG. 3 shows a semi-transparent view of a breath control training configuration of a modular oral motor training system, according to an embodiment of the present invention;

FIG. 4 schematically illustrates an exemplary configuration of a modular oral motor training system, according to an embodiment of the present invention;

FIG. 5 shows a semi-transparent view of tongue thrust training device, according to an embodiment of the present invention;

FIG. 6 shows a semi-transparent view of a suction training device, according to an embodiment of the present invention;

FIG. 7 shows a semi-transparent view of a mastication training device, according to an embodiment of the present invention; and

FIG. 8 shows a semi-transparent view of a sound production training device, according to an embodiment of the present invention.

### DETAILED DESCRIPTION OF THE INVENTION

The present invention relates to a modular oral motor training system adapted to operate with multiple electromechanical training devices to strengthen the oral musculature, and improve the oral motor and breathe control, thereby improving the speech fluency and pronunciation of persons challenged with speech disorders.

The modular system of the invention comprises a base unit onto which multiple interchangeable functional training devices are detachably attached, each of which is adapted with an operable mechanism (i.e., designated to be mechanically operated by a user). The system comprises suitable control and monitoring means for controlling the operable mechanism and/or for assessing its operation by a user of the proposed system by converting a mechanical load applied by the user to an electrical signal which is coded by suitable processing unit into a performance level (e.g., using an airflow sensor which converts the speed of blown air into an air-blow training device, into an electrical signal. The signal is transferred to a basic controller adapted to receive the signal from the sensor and to code it into a performance level). The performance level is transmitted in turn to the base unit, which provides performance feedback.

In the following detailed description, references are made to the accompanying drawings that form a part hereof and shown by way of illustrating specific embodiments or examples. These embodiments may be combined, other embodiments may be utilized, and other changes may be made without departing from the spirit or scope of the present invention.

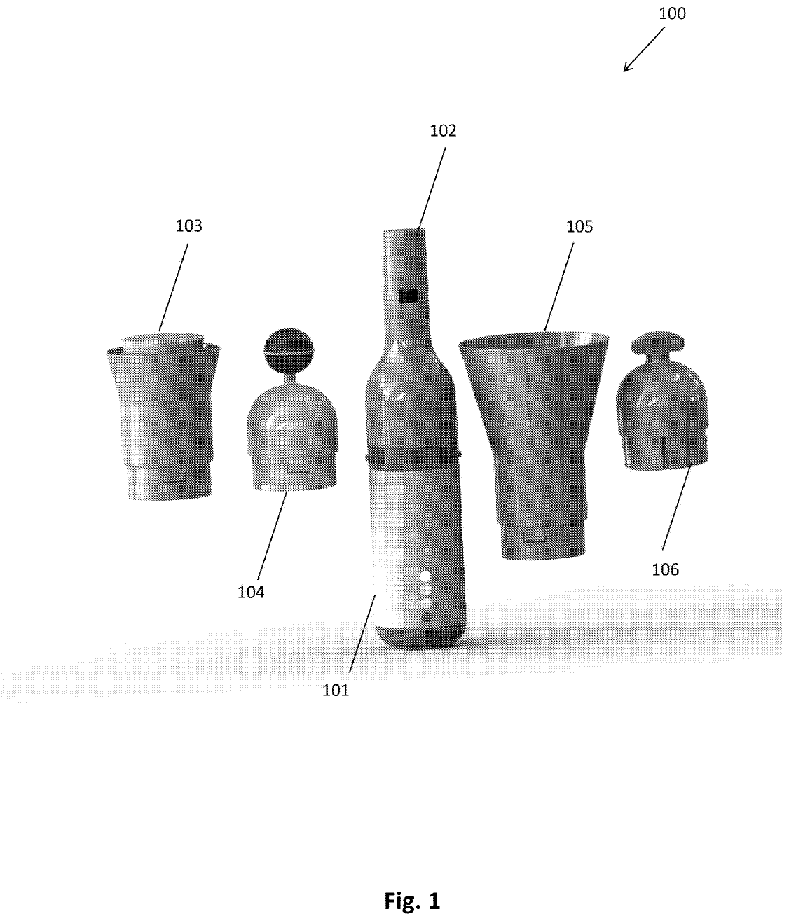

FIG. 1 shows an exemplary configuration of a modular oral motor training system, according to an embodiment of the present invention. System 100 comprises a base unit 101, an air-blow training device 102, a tongue thrust device 103, a suction device 104, a mastication training device 105, and a sound production training device 106. Of course, alternate functional training devices may be adapted to pair with base unit 101, to interface with the electromechanical coupling means of the base unit 101, as will be illustrated by the following description.

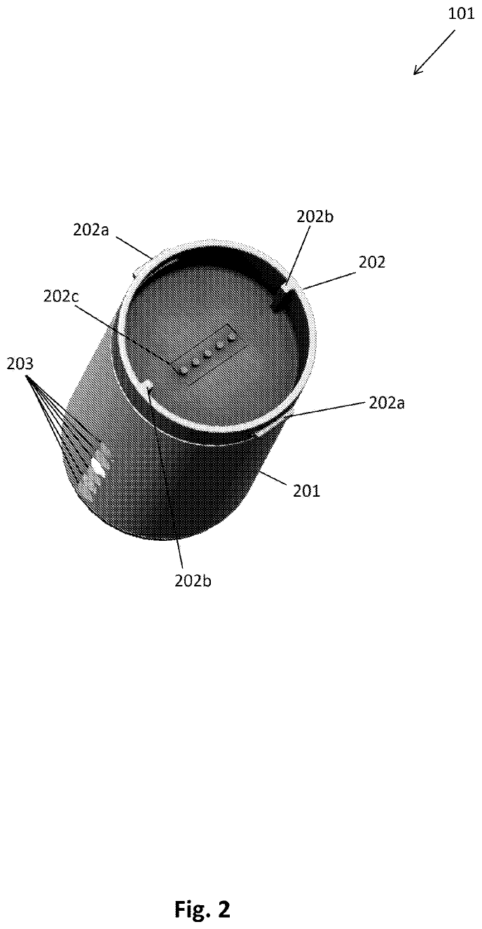

FIG. 2 shows a base unit 101, according to an embodiment of the present invention. The base unit 101 of training system 200 comprises an enclosure 201 (e.g., of a cylindrical shape) used for holding system 200, and an electromechanical coupling port 202. Accoridn to an embodiment of the invention, electromechanical coupling port 202 comprises a mechanical coupling arrangement (e.g., spring-loaded clamps 202a and orientation protruding 202b) suitable for providing a secured and properly oriented coupling of a training device (102-106) with the base unit 101. Coupling port 202 further comprises electric connector 202c, such as a spring-loaded-pins connector (e.g., Pogo Pins connector), being utilized for power supply and/or data transfer between a paired training device (102-106) and the base unit 101.

The base unit 101 further comprises one or more audial and/or visual feedback means which produce real-time feedback corresponding to the performance of a user of system 200, such as Light Emitting Diodes (LEDs) 203, which can produce light of various predetermined colors and flash at controllable frequency and order (e.g., turning more/fewer LEDs 203 in accordance with the user's performance in a current functional task such as stronger air-blowing into air-blow training device 102 of FIG. 1). The feedback form and intensity (e.g., the sound of predetermined frequency and amplitude, light of predetermined color, and flashing frequency) may be configured to reflect training difficulty and users' performance. Thereby providing performance feedback adapted to a user's characteristic (e.g., soft animal sounds for younger children, audial sentences for older children). According to some embodiments of the present invention, base unit 101 comprises an oscillator for producing desirable vibrations as performance feedback.

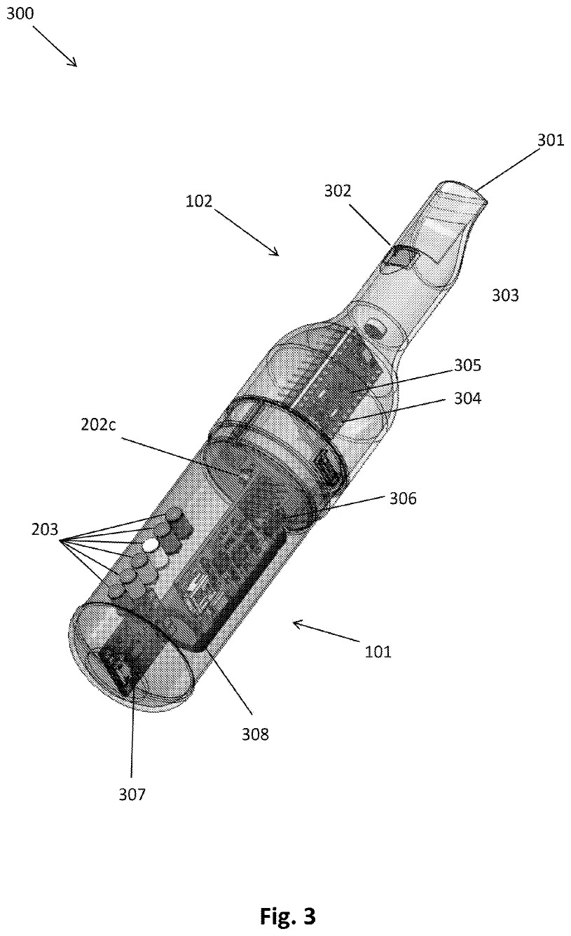

FIG. 3 shows a partial transparent view of a breathe control training configuration of a modular oral motor training system, according to an embodiment of the present invention, in which system 300 comprises base unit 101 coupled with air-blow training device 102 (i.e., by corresponding electromechanical coupling means to those of coupling port 202 of base unit 101), wherein air-blow training device 102 comprises a mouthpiece 301 into which a user blows air (i.e., thereby exercising multiple involved muscles, such as the diaphragm, chest muscles, facial muscles, and masticatory system), an air outlet 302 through which the blown air flows out, and an air flow sensor 303 (e.g., a pressure or velocity sensor) which is adapted to detect air capacity data (e.g., of air flowing in-between mouthpiece 301 and outlet 302) and to transmit a corresponding electrical signal to an electronic circuitry module such as a Printed Circuit Board (PCB) 304 comprising a controller 305 for processing the electrical signal. For example, the air capacity data is coded into a performance level by the electronic circuitry module, and transmitted to base unit 101 for producing a suitable feedback.

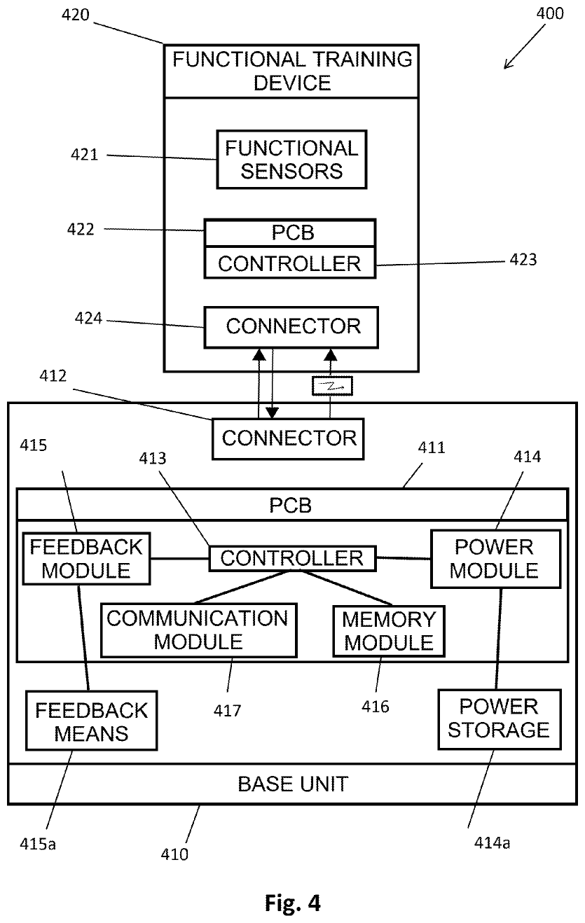

FIG. 3 further shows an internal view of the base unit 101, showing a PCB 306, which may comprise the suitable hardware for managing system 300 (e.g., main controller 306a and other components detailed in FIG. 4). PCB 306 mechanically supports and electrically connects electrical or electronic components, such as connector 202c and a charging module 307 (e.g., integrated USB charging module). For example, charging module 307 may control the charging of a rechargeable power storage element, such as a rechargeable lithium battery 308, which supplies the required power for operating the electrical circuitry of air-blow training device 202 through connector 202c.

FIG. 4 schematically illustrates an exemplary configuration of a modular oral motor training system, according to an embodiment of the present invention, in which system 400 comprises a base unit 410 and a functional training device 420, wherein base unit 410 comprises a PCB 411, and a connector 412 which provides data and power transfer to a functional training device 420, where PCB 411 comprises a main controller 413 which is adapted to control and to monitor the operation of system 400 (e.g., the power supply to device 420 through connectors 412 and 424 which can be triggered upon proper coupling of device 420 with base unit 410 and/or by a power button (not shown) installed in unit 410), a power module 414 which manages a power storage component 414a (e.g., a disposable battery, rechargeable battery 308 and charging module 307), a feedback module 415 which controls feedback means 415a (e.g., LEDs 203), a memory module 416, which stores previous training data, and a wired/wireless communication module 417 (e.g., utilizing a USB port for wired data transfer, a Bluetooth device for short range wireless data connection, WIFI device, etc.), which is utilized for transferring the training and performance data to a remote computing device (e.g., a smartphone, a local computer, a remote server, etc.).

According to some other embodiments of the present invention, the communication means are utilized to upload clinical data (e.g., user's performance in various training mode). The clinical data enables a remote training personalization for each use and a collection of mass clinical data to be processed by a central computing device running machine learning protocols/algorithms for continuously studying and improving the training programs.

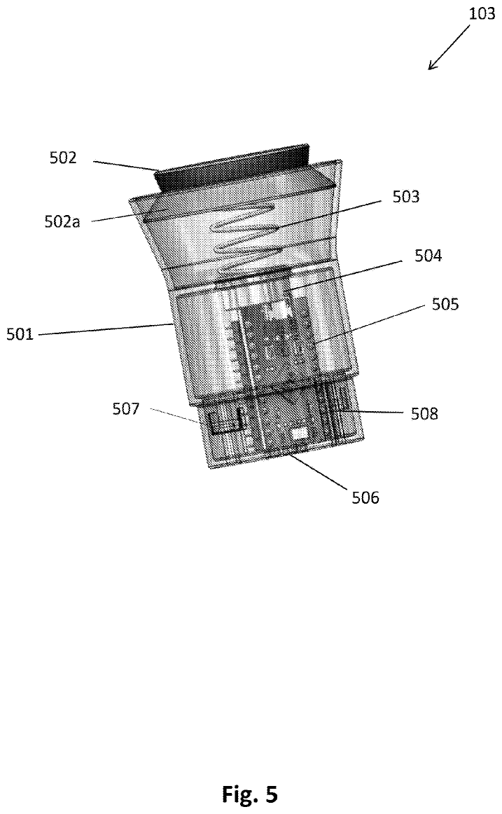

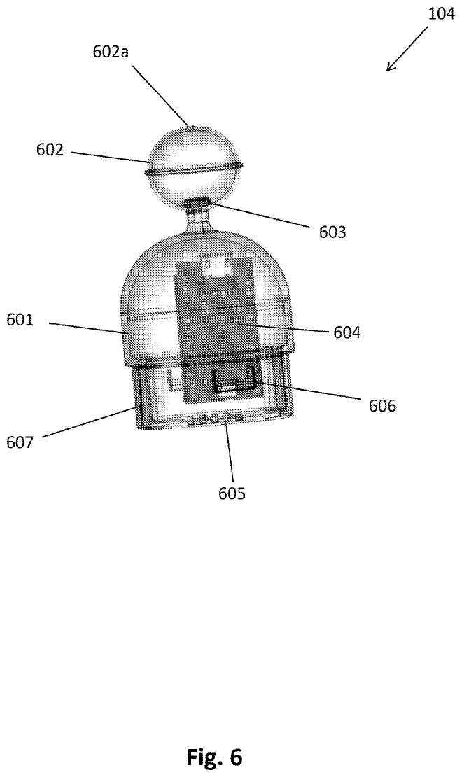

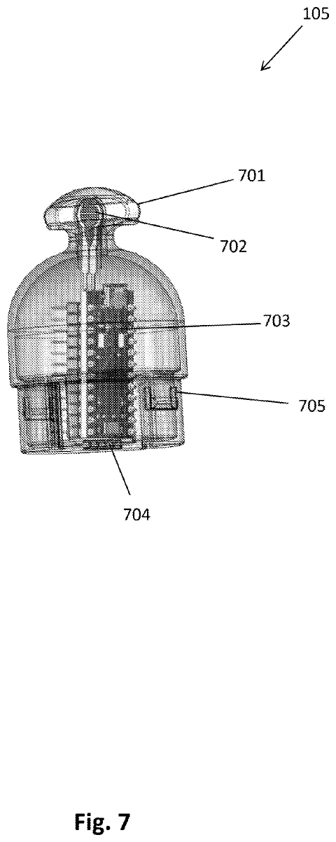

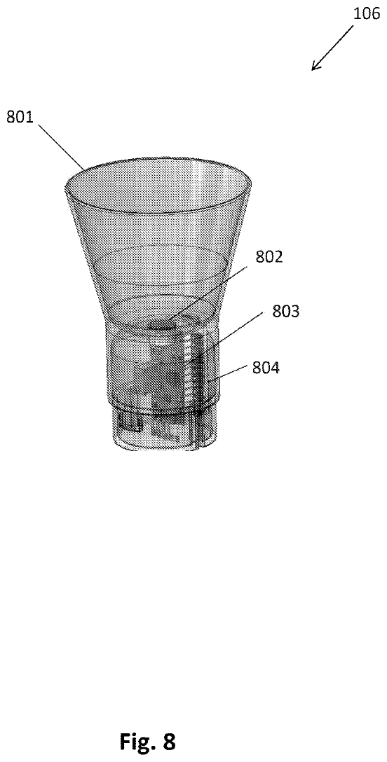

Functional training device 420 may be any suitable oral motor training device (e.g., air-blow training device 102 of FIG. 3, tongue thrust device 103 of FIG. 5, suction device 104 of FIG. 6, mastication training device 105 of FIG. 7, sound production training device 106 of FIG. 8, etc.) adapted to connect with a base unit 410 (e.g., such as base unit 101 of FIGS. 1-3) with corresponding electromechanical means to port 202 of the base unit 101. Each functional training device 420, essentially comprises an operable mechanism (shown in FIGS. 3 and 5-8), one or more functional sensors 421 which detect a user's performance and transfers corresponding electrical signals to a PCB 422 comprising a controller 423 which inter alia codes the received electrical signals into a performance level, and a connector 424, through which the functional training device 420 receives power (e.g., by dedicated pins of corresponding connectors 412 and 424) and communicates with the base unit 410 (e.g., transmitting the performance level to PCB 411).

FIG. 5 shows a semi-transparent view of tongue thrust training device 103 enclosed within an essentially cylindrical enclosure 501, according to an embodiment of the present invention. Training device 103 comprises a thrust surface 502 supported by a resisting spring 503 (i.e., of a known constant) which engages a fixed pressure sensor 504. When surface 501 is pressed by a user's tongue (i.e., the user exercises the muscles of the tongue, which are essential for proper pronunciation), the thrust load is transferred through spring 502 to sensor 504, which transmits a corresponding electrical signal to PCB 505. The electrical signal amplitude is coded into a performance level, and transmitted to a base unit 410 (e.g., the base unit 101) for producing suitable feedback. Further shown in FIG. 5 is a silicone gasket 502a which prevents the penetrating of humidly into enclosure 501, a connector 506 (i.e., corresponding with connector 202a) through which training device 103 receives power and communicates with the base unit 101, flexible protrusions 507 (i.e., corresponding with clamps 202a of the base unit 101), and grooves 508 which are adapted to engage orientation protruding 202b (shown in FIG. 2) of the base unit 101, thereby ensuring properly oriented coupling of tongue thrust training device 103 with the base unit 101.

FIG. 6 shows a semi-transparent view of a suction training device 104 enclosed within an essentially cylindrical enclosure 601, according to an embodiment of the present invention. Training device 104 comprises a vacuum ball 602 with an orifice 602a, through which suction vacuum is exerted by a user (i.e., the user exercises the rear-tongue muscles) into the cavity of ball 602 and is detected by a pressure sensor 603, which transmits the corresponding electrical signal to an electronic circuitry module such as a PCB 604 for processing the signal. For example, the signal amplitude is coded by the electronic circuitry module into a performance level, which is transmitted to a base unit 410 (e.g., the base unit 101) for producing a feedback. Training device 104 further comprises a connector 605 through which training device 104 receives power and communicates with the base unit 101. Training device 104 may further comprise mechanical attachment means adapted to physically engage the base unit 101. In this embodiment, the mechanical attachment means comprise flexible protrusions 606 (i.e., adapted to fit with clamps 202a of the base unit 101 of FIG. 1), and grooves 607, which are adapted to engage orientation protruding 202b (shown in FIG. 2) of the base unit 101, thereby ensuring properly oriented coupling of suction training device 104 with the base unit 101.

FIG. 7 shows a semi-transparent view of a mastication training device 105, according to an embodiment of the present invention, in which training device 105 comprises a biting mouthpiece 701, onto a biting load is applied by a user's teeth (i.e., the user exercises the mastication muscles), and is detected by a force-sensitive sensor 702 which transmits the corresponding electrical signal to a PCB 703, in which the signal amplitude is coded into a performance level, which is transmitted to a base unit 410 (e.g., base unit 101) for producing suitable feedback. Training device 105 further comprises a connector 704 through which training device 105 receives power and communicates with the base unit 101, and flexible protrusions 605 (i.e., corresponding with clamps 202a of the base unit 101 of FIG. 1) for ensuring properly secured coupling of mastication training device 105 with the base unit 101.

FIG. 8 shows a semi-transparent view of a sound production training device 106, according to an embodiment of the present invention, in which training device 106 comprises a resonance cavity 801 into a user produces sound (i.e., the user exercises the diaphragm, the chest muscles, masticatory system, and the phonatory muscles), of which pitch and amplitude are detected by a sound sensor 802 in connection with voice recognition module 803, which transmits the corresponding electrical signal to a PCB 804, in which the signal amplitude is coded into a performance level, which is transmitted to a base unit 410 (e.g., the base unit 101) for producing suitable feedback.

The modular design of the proposed system, as described in FIGS. 1-8, enables an effortless attachment/detachment of adapted functional training devices to a base unit such as base unit 101 of FIGS. 1-3. Moreover, the illustrated training devices 102-106 are of a simplistic design, enabling easy maintenance and cleaning of the training devices.

Although embodiments of the invention have been described by way of illustration, it will be understood that the invention may be carried out with many variations, modifications, and adaptations without exceeding the scope of the claims. For example, the base unit 101 may be provided with an ergonomic holding element such as an externally hex-shaped enclosure 201 or with an ergonomic silicone ring threaded onto enclosure 201.

1. A modular oral motor training system, comprising a base unit adapted to receive one or more replaceable functional training devices, wherein said base unit comprises an electromechanical port through which said base unit supplies electrical power and receives performance level data to/from said one or more functional training devices, according to which said base unit delivers performance feedback to a user of said modular oral motor training system.

2. The system of claim 1, in which each of the one or more functional training devices comprises an operable mechanism, suitable means for assessing its mechanical operation by a user, suitable control and processing hardware for coding the assessed mechanical operation of said operable mechanism by the user into a performance level, and electromechanical means for coupling said one or more functional training devices with said coupling electromechanical port of said base unit, wherein said performance level is transmitted to said base unit for producing suitable feedback.

3. The system of claim 1, in which the delivered feedback is selected from the group consisting of: audial feedback, visual feedback, mechanical feedback, or any combination thereof.

4. The system of claim 1, in which the electromechanical port comprises a Pogo Pins connector.

5. The system of claim 1, in which the base unit is cylindrical.

6. The system of claim 1, in which the base unit comprises a communication module.

7. The system of claim 1, in which the one or more functional training devices are selected from the group consisting of: an air-blow training device, a tongue thrust device, a suction device, a mastication training device, a sound production training device, or any combination thereof.
